(19) United States
(12) Patent Application Publication
(10) Pub. No.: US 2026/0157744 A1
(43) Pub. Date: Jun. 11, 2026

(54) Driver to Eyelet Attachment and Methods of Tissue Repairs

(71) Applicant: Arthrex, Inc., Naples, FL (US)

(72) Inventors: Jacob R. CHARLES, Cape Coral, FL (US); Brittany R. FOSLIEN, Naples, FL (US); Phillip D. CHEE, Naples, FL (US)

(73) Assignee: Arthrex, Inc., Naples, FL (US)

(21) Appl. No.: 19/367,142

(22) Filed: Oct. 23, 2025

Related U.S. Application Data
(60) Provisional application No. 63/730,679, filed on Dec. 11, 2024.

(51) Int. Cl. A61B 17/04; A61F 2/08

(52) U.S. Cl. CPC A61B 17/0401; A61B 17/0466; A61F 2/0811; A61B 2017/0409; A61B 2017/0414; A61B 2017/0445; A61B 2017/0464; A61F 2002/0841; A61F 2002/0852; A61F 2002/0882

(57) ABSTRACT

Surgical constructs, assemblies and methods for surgical repairs are disclosed. A swivel anchor is employed with a driver provided with a driver-eyelet attachment mechanism that allows a suture eyelet to engage a mating driver shaft. The attachment mechanism includes one or more flexible prongs on the driver tip that are configured to mate with one or more apertures on the eyelet. The prongs are configured to flex in and out of the eyelet apertures. The swivel anchor may be locked and secured within tissue by inserting a cannulated fixation device to abut and contact the eyelet.

### CROSS-REFERENCE TO RELATED APPLICATIONS

This application claims priority to U.S. Provisional Application No. 63/730,679 filed Dec. 11, 2024, the disclosure of which is hereby incorporated by reference in its entirety herein.

### BACKGROUND

The disclosure relates to the field of surgery and, more specifically, to anchor constructs, assemblies and associated methods of tissue repairs.

### SUMMARY

Surgical systems, assemblies, and methods for tissue fixation are disclosed.

A surgical assembly includes an anchor for knotless tissue fixation. An anchor may be employed with a driver provided with an attachment mechanism that allows a suture eyelet to engage a mating driver shaft. An attachment mechanism may include one or more prongs on the driver tip that are configured to mate with one or more apertures on the eyelet. The prongs may be configured to flex in and out of the eyelet apertures. In some implementations, the anchor may be a swivel anchor. In some implementations, a swivel anchor may be locked and secured within tissue by inserting a cannulated fixation device to contact the eyelet.

A method for knotless fixation of anatomical tissue during surgical applications may include employing an anchor assembly having a driver-eyelet attachment mechanism. The method may include: (i) capturing at least one flexible strand with an eyelet; (ii) engaging a driver tip to the eyelet with a driver-eyelet attachment mechanism; (iii) securing the eyelet with the at least one flexible strand into hard tissue. The eyelet may be secured into hard tissue by providing a cannulated fixation device to contact the eyelet. The anchor may be a swivel anchor.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a driver of an anchor assembly.

FIG. 2 illustrates an eyelet of an anchor assembly.

FIG. 3 illustrates another eyelet of an anchor assembly.

FIG. 4 illustrates a surgical assembly with the eyelet of FIG. 2 engaging the driver of FIG. 1.

FIG. 5 illustrates the surgical assembly of FIG. 4 at a stage subsequent to that of FIG. 4.

FIG. 6 illustrates the surgical assembly of FIG. 5 at a stage subsequent to that of FIG. 5.

FIG. 7 illustrates the surgical assembly of FIG. 6 at a stage subsequent to that of FIG. 6.

FIG. 8 illustrates the surgical assembly of FIG. 7 with suture threaded through the eyelet.

FIG. 9 illustrates the surgical assembly of FIG. 8 at a stage subsequent to that of FIG. 8.

FIG. 10 illustrates the surgical assembly of FIG. 9 at a stage subsequent to that of FIG. 9.

FIG. 11 illustrates the surgical assembly of FIG. 10 at a stage subsequent to that of FIG. 10.

FIGS. 12-14 illustrate an exemplary repair with the surgical assembly of FIG. 8.

### DETAILED DESCRIPTION

Assemblies, apparatus and methods for tissue fixation using suture anchors are disclosed. In some implementations, a swivel anchor may be used with a driver configured to securely engage and retain an eyelet carrying one or more flexible strands. A swivel anchor driver-eyelet attachment mechanism allows an eyelet to be retained on a driver tip and rotate freely on the driver tip during anchor insertion. The eyelet disengages the driver tip when the swivel anchor is fully inserted. In some implementations, the anchor pushes the eyelet off. In some implementations, the eyelet pops off the driver tip. In some implementations, one or more flexible strands from the eyelet extend outside the driver shaft. In some implementations, any and/or all flexible strands from the eyelet extend outside the driver shaft. The eyelet may rotate to about 360 degrees while preventing suture from twisting around the driver during anchor insertion and from being tangled.

The driver shaft is provided with a tip that mates with the eyelet. In some implementations, the driver tip may include one or more prongs that flex in and out. In some implementations, the driver tip may include two prongs that flex in and out. The mating eyelet is provided with corresponding windows or apertures to engage the prongs and retain the eyelet on the driver tip. In some implementations, the prongs flex in when the eyelet is pushed onto the driver tip and flex out when the eyelet is fully seated on the driver tip. When the swivel anchor is pushed up against the eyelet, the prongs flex in and the eyelet is pushed off the driver tip.

At least one flexible strand is passed through the eyelet on the driver. The driver may be also used to engage and lock the eyelet in a hole in bone by inserting a cannulated fixation device (for example, an anchor, an interference plug or screw, or an implant) to contact the eyelet.

A surgical assembly may include a suture anchor for knotted or knotless tissue fixation. In some implementations, the anchor may be a swivel anchor. In some implementations, the anchor may be a knotless swivel anchor. In some implementations, a swivel anchor may be employed with a driver provided with an attachment mechanism that allows a suture eyelet to engage the driver. An attachment mechanism may include one or more prongs that are provided on the driver tip and are configured to mate with one or more windows, apertures or openings on a proximal end of the eyelet. The prongs may be configured to flex in and out of the eyelet apertures.

In some implementations, the driver may be solid and the flexible strand(s) such as suture(s) extend along an outer surface of the driver. A swivel anchor may be locked and secured within tissue by inserting a cannulated fixation device (for example, an anchor, an interference plug or screw, or an implant) to abut and contact the eyelet.

Methods of tissue repairs are also disclosed. A method for fixation of anatomical tissue during surgical applications may include employing an anchor assembly provided with a driver-eyelet attachment mechanism. The method may include: (i) capturing at least one flexible strand with an eyelet; (ii) attaching a driver tip to the eyelet by engaging a driver-eyelet attachment mechanism; and (iii) securing the eyelet with the at least one flexible strand into tissue. The eyelet may be secured into tissue by providing a cannulated fixation device (for example, an anchor, an interference plug or screw, or an implant) to abut and contact the eyelet. The eyelet and cannulated fixation device may form and be part of a swivel anchor.

A method for knotless fixation of anatomical tissue during surgical applications by employing a suture anchor and a driver-eyelet attachment mechanism may include the steps of: (i) inserting an eyelet on a mating driver shaft by engaging an attachment mechanism; and (ii) securing the eyelet with an anchor within a bone hole by disengaging the attachment mechanism. The attachment mechanism may include one or more prongs on the driver shaft configured to mate with one or more windows or apertures on the eyelet. The prongs may be configured to flex in and out of the eyelet apertures.

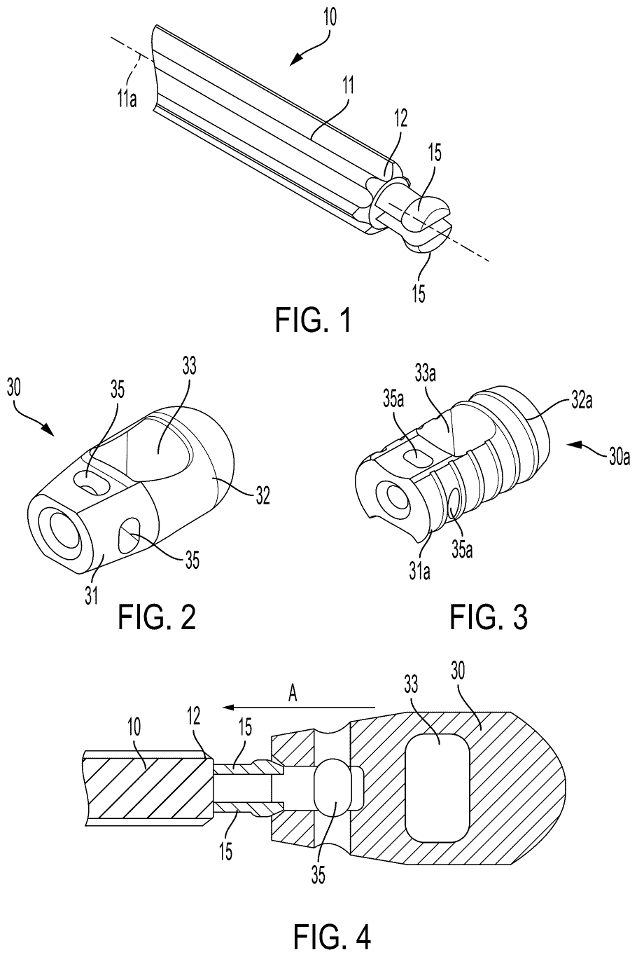

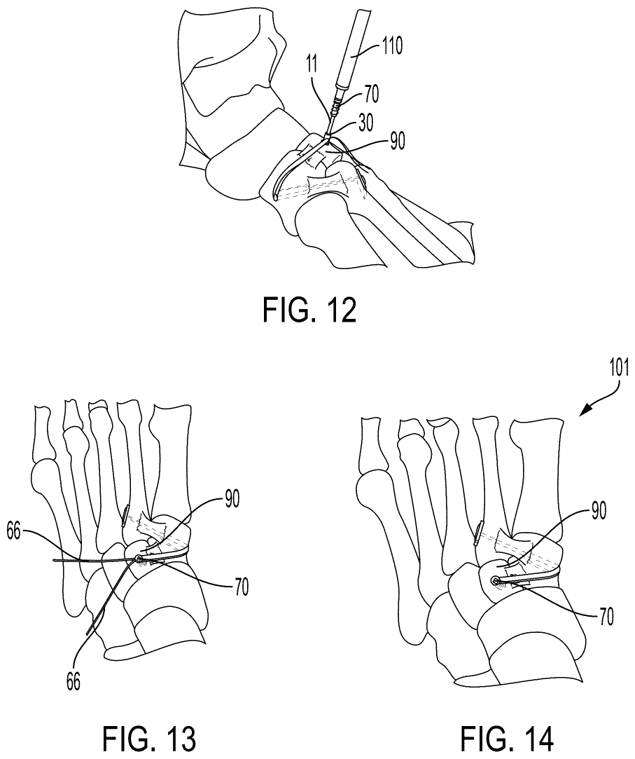

Referring now to the drawings, where like elements are designated by like reference numerals, FIGS. 1-11 illustrate various structural elements of surgical assembly 110 and anchor 100. Anchor 100 may be a swivel anchor; a suture anchor; an implantable device; an implant; an anchor assembly; an anchor system; a tensionable, knotless construct; or a SwiveLock® anchor. FIGS. 12-14 illustrate an exemplary repair 101 with surgical assembly 110 and anchor 100.

Reference is now made to FIGS. 1-8, which illustrate details of driver assembly 110 (FIG. 8) with driver 10 and anchor 100. Driver 10 is a solid, non-cannulated driver including shaft 11 and an inserter handle (not shown). One or more prongs 15 are provided at a most distal end 12 of driver 10. In some implementations, the prongs may be flexible members, projections, tines, tabs or protuberances designed to engage and fit into apertures or windows provided within a proximal end of eyelet 30 of anchor 100, and as detailed below.

In some implementations, and as illustrated in FIG. 1, two prongs 15 are provided symmetrically positioned relative to longitudinal axis 11a of driver shaft 11 and extending away from the most distal end 12. Although the embodiments below will be described with reference to driver assembly 110 having two exemplary prongs 15, it must be understood that the disclosure is not limited to this exemplary-only implementation and contemplates any number of prongs extending away from the distal end 12 of shaft 11. Prongs 15 may extend about parallel to longitudinal axis 11a of shaft 11 and may have similar configurations and dimensions (as shown in FIG. 1) or may have different configurations and dimensions. In some implementations, prongs 15 may consist of a plurality of distal radially expanding projections that are designed to pass through corresponding windows/openings/apertures in a proximal portion of the eyelet 30. Prongs 15 are configured to move in at least an inward direction and an outward direction. Preferably, the windows/openings in the proximal portion of the eyelet 30 have a shape and geometry that is complementary to that of the prongs 15.

FIGS. 2 and 3 illustrate perspective views of two exemplary embodiments of anchor eyelet 30 (FIGS. 2) and 30a (FIG. 3) that are configured to engage prongs 15 of driver 10 and retain at least one flexible strand. Eyelet 30 of FIG. 2 is detachable and provided with a body that includes a distal tip 32 and a transverse through-hole or suture opening 33 (suture eyelet 33) to allow passage of at least one flexible strand (suture) through it. Distal tip 32 may be rounded (as shown in FIG. 2) or may have a pointed configuration to help with insertion of the anchor eyelet within a bone hole or opening. A plurality of windows/through-holes/openings/slots/apertures 35 are provided at proximal end 31 of eyelet 30, adjacent the suture opening 33 without intersecting the suture opening 33. In some implementations, two windows/through-holes/openings/slots/apertures 35 are provided within the anchor eyelet 30 to allow engagement with prongs 15. Preferably, the windows/through-holes/openings/slots/apertures in proximal portion 31 of the eyelet 30 have a shape and geometry that is complementary to that of the prongs 15 at the driver tip. In this manner, when eyelet 30 is inserted into the driver shaft, the prongs 15 expand radially outwardly and pass through (through lateral movement, for example) the windows 35 provided in the eyelet 30. The prongs 55 are designed to engage the inner surface of the windows 35 to prevent accidental separation of the driver shaft from the eyelet.

Eyelet 30a of FIG. 3 is detachable and provided with a body that includes a distal tip 32a with a most distal surface having about a flat configuration and with a transverse through-hole or suture opening 33a (suture eyelet 33a) to allow passage of at least one flexible strand (suture) through it. A plurality of windows/through-holes/openings/slots/apertures 35a are provided at proximal end 31a and adjacent the suture opening 33a without intersecting the suture opening 33a. In some implementations, two windows/through-holes/openings/slots/apertures 35a are provided within the anchor eyelet 30a to allow engagement with prongs 15. Outer surface of eyelet 30a may be ribbed (as shown in FIG. 3) to allow better engagement of bone during anchor eyelet insertion within a bone hole or opening.

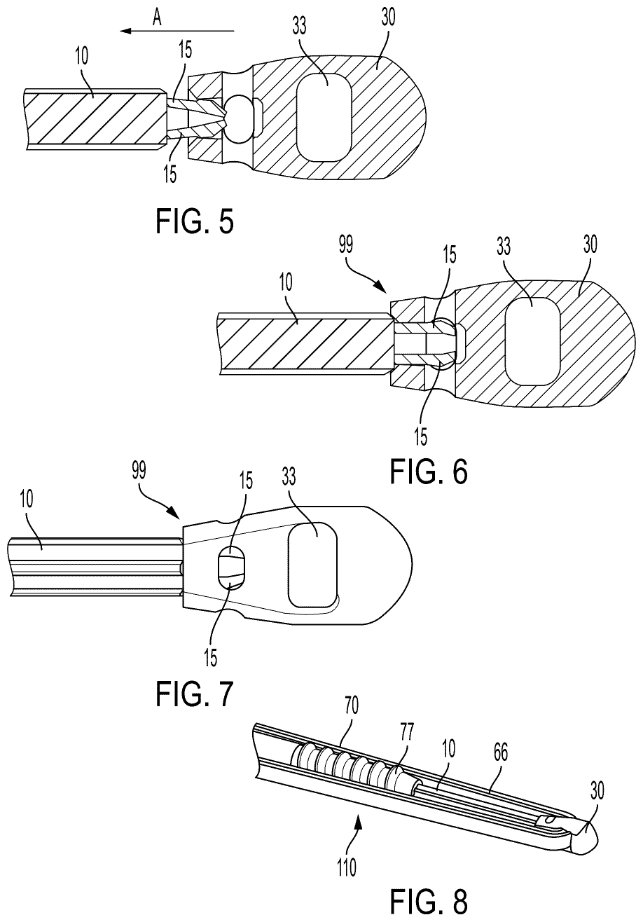

Reference is now made to FIGS. 4-8, which illustrate engagement of driver 10 of FIG. 1 with eyelet 30 of FIG. 2 to form attachment mechanism 99 of anchor driver assembly 110:
FIG. 4: Insert eyelet 30 on mating driver shaft 11 in the direction of arrow A.
FIG. 5: The prongs 15 on the driver shaft 11 temporarily bend when pushing eyelet 30 on. The bending of the prongs 15 is inwardly relative to the eyelet 30.
FIGS. 6 and 7: Prongs 15 on driver shaft 11 spring back to original shape once eyelet 30 is fully inserted. FIG. 6 is a cross-sectional view of the distal end of attachment mechanism 99; FIG. 7 is a side view of the distal end of attachment mechanism 99.
FIG. 8: One or more flexible strands 66 are passed through suture eyelet 33 of eyelet 30 and extending on the outer surface of solid shaft 11 of driver 10. Flexible strand(s) 66 may include any flexible coupler and may be formed of various flexible materials and strands such as round suture, flat suture, ribbon, or flat tape (for example, suture tape) or combination of suture and tape, among many others.

FIG. 8 also illustrates exemplary fixation device 70 that is employed in conjunction with the driver assembly 110 and anchor 100. The fixation device 70 may be preloaded on the driver assembly 110. Fixation device 70 may be a cannulated fixation device such as, for example, an anchor, an interference plug or screw, or an implant. As detailed below, cannulated fixation device 70 (anchor 70) is advanced by turning the driver assembly to which the anchor 70 will start contacting the eyelet 30. Anchor 70 is provided with a cannulated body having an outer surface provided with one or more fixation structures 77 (such as threads 77, for example) for securing the anchor body in a bone hole. The assembly is ready for use.

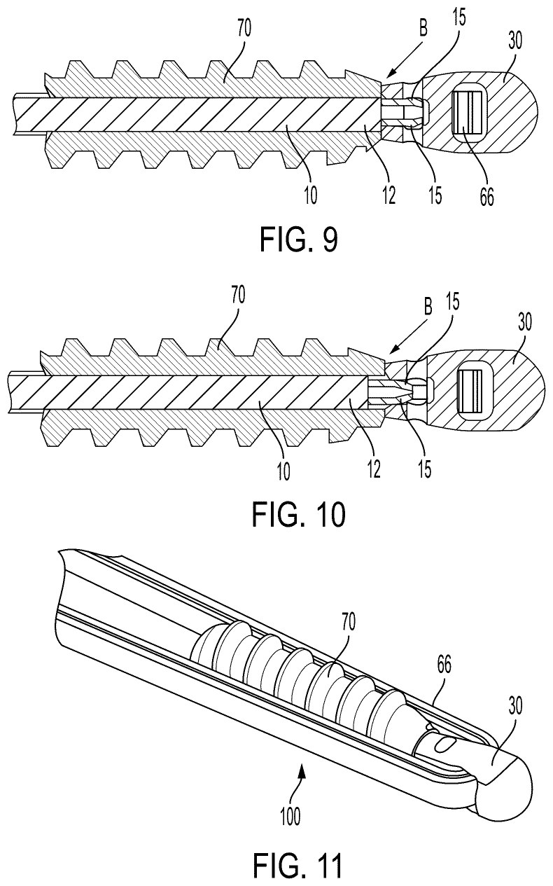

Reference is now made to FIGS. 9-11, which illustrate disengagement of driver 10 from eyelet 30 to allow anchor 100 (FIG. 11) to be fully seated within a bone hole or opening:
FIG. 9: Surgeon prepares the applicable bone and inserts the eyelet 30 into hole. Anchor 70 is advanced by turning the driver assembly to which the anchor 70 will start contacting the eyelet 30 at position or location B.
FIG. 10: Surgeon continues to advance the anchor 70 causing the anchor 70 to push the eyelet 30 off the prongs 15. Attachment mechanism 99 is disengaged allowing the driver 10 to be removed from eyelet 30 and from the surgical site.
FIG. 11 depicts the entire construct once fully inserted. Anchor 100 may be a swivel anchor 100. Anchor 100 includes eyelet 30 and cannulated fixation device 70 (anchor 70). At least one flexible strand 66 passes through suture eyelet 33 of eyelet 30, extending on an outer surface of driver shaft 11.

In some implementations, the fixation device 70 may be advanced into the bone socket by holding a thumb pad as the driver/inserter handle is turned clockwise. When the fixation device 70 is fully seated, the most distal end of the fixation device 70 abuts and contacts the most proximal end of eyelet 30 to optimize the stability of the swivel anchor construct 100. As previously stated, the swivel anchor construct 100 is composed of eyelet 30 and fixation device 70.

In some implementations, anchor construct 100 may be a swivel anchor about similar to a SwiveLock® anchor. The SwiveLock® driver tip 12 has two prongs 15 that flex in and out, and the mating eyelet 30 has holes 35 to retain the eyelet 30 on the driver tip 12. The two prongs 15 flex in when the eyelet 30 is pushed onto the driver tip 12 and the prongs 15 flex out when the eyelet 30 is fully seated on the driver tip 12. When the SwiveLock® anchor 70 is pushed up against the eyelet 30, the two prongs 15 flex in and the eyelet 30 is pushed off the driver tip 12. The SwiveLock® driver-eyelet attachment mechanism 99 allows the eyelet 30 to be retained on the driver tip 12 and rotate freely on the driver tip 12 during anchor insertion. The eyelet 30 pops off the driver tip 12 when the SwiveLock® anchor 100 is fully inserted (the anchor 70 pushes the eyelet off).

The swivel anchor design of the present disclosure overcomes drawbacks of the prior art designs which have eyelets that are threaded onto the driver tip and are difficult to pop off or that may cause suture to twist around the driver during anchor insertion. The swivel anchor design of the present disclosure enables rotational insertion of the suture anchor without causing excessive twisting and knotting of the flexible strand 66 by eyelet 30.

Flexible strand 66 may be passed through the tissue and can be secured either using a single anchor (such as anchor 100) or a plurality of anchors. In addition, various anchors, such as those noted above and others, may be used interchangeably with only slight variations in the procedure.

In some implementations, anchor 100 detailed above may be employed with additional independent fixation devices such as, for example, knotted anchors, knotless anchors, or all-suture anchors, or any devices that confer secure attachment and fixation of first tissue to a second tissue. Additional fixation devices that may be used with anchor 100 and assembly 110 are, for example, knotless anchors such as a two-piece Arthrex PushLock® anchor, disclosed in U.S. Pat. No. 7,329,272, or Arthrex SwiveLock® anchors, disclosed in U.S. Pat. Nos. 8,012,174 and 9,005,246, the disclosures of both of which are fully incorporated by reference in their entirety herein.

Reference is now made to FIGS. 12-14 which illustrate an exemplary tissue repair 101 with anchor assembly 100. Repair 101 is an exemplary small extremities repair (such as a Lisfranc fixation system for InternalBrace™ repair) which may employ one or more anchors 100 of the present disclosure.

As shown in FIG. 12, two flexible strands 66 (for example, two FiberTape® suture tails) are loaded through anchor 100. In some implementations, anchor 100 may be an exemplary 3.5 mm×13.5 mm swivel anchor 100. Anchor 100 is inserted into a pre-formed hole in the central portion of intermediate cuneiform 90. Strands 66 (FIG. 13) can be trimmed and cut. Final fixation of repair 101 is shown in FIG. 14.

The constructs, systems, and assemblies of the present disclosure may be employed in various soft tissue repairs and fixations, for example, fixation of soft tissue to bone and for any tissue positioning and/or tissue adjustment applications. The driver-eyelet attachment mechanism, assemblies and anchors of the present disclosure may be utilized in surgical procedures such as rotator cuff repair, Achilles tendon repair, patellar tendon repair, ACL/PCL reconstruction, hip and shoulder reconstruction procedures, AC joint reconstruction, syndesmosis reconstruction, quad/patellar tendon rupture repair, hallux-valgus repair, proximal and/or distal biceps tendon repair, humerus and radius repair, and any other tendon repair to bone, among many others. These repairs may be conducted in a knotless manner.

A surgical assembly 110 may include a suture anchor 100 for knotted or knotless tissue fixation. In some implementations, the anchor 100 may be a swivel anchor. In some implementations, a swivel anchor may be employed with a driver 10 provided with a driver-eyelet attachment mechanism 99 that allows an eyelet 30 with suture 66 to engage the driver 10. An attachment mechanism 99 may include one or more prongs 15 that are provided on the driver tip 12 and are configured to mate with one or more windows or apertures 35 on the eyelet 30. The prongs 15 are configured to flex in and out of the eyelet apertures 35. Driver 10 may be solid and the flexible strand(s) 66 may extend along an outer surface of the driver 10. A swivel anchor 100 may be locked and secured within tissue by inserting a cannulated fixation device 70 (for example, an anchor, an interference plug or screw, or an implant) to contact and abut the eyelet 30.

Methods of tissue repairs are also disclosed. A method for fixation of anatomical tissue during surgical applications may include employing an anchor driver assembly 110 provided with a driver-eyelet attachment mechanism 99. The method may include: (i) capturing at least one flexible strand 66 with eyelet 30; (ii) attaching a driver tip 12 to the eyelet 30 by engaging a driver-eyelet attachment mechanism 99; and (iii) securing (implanting) the eyelet 30 with the at least one flexible strand 66 into hard tissue. The eyelet 30 may be secured into hard tissue by providing a cannulated fixation device 70 (for example, an anchor, an interference plug or screw, or an implant) to abut the eyelet 30. The eyelet 30 and cannulated fixation device 70 form swivel anchor 100.

A method for knotless fixation of anatomical tissue during surgical applications by employing a suture anchor 100 and a driver-eyelet attachment mechanism 99 may include the steps of: (i) inserting an eyelet 30 on a mating driver shaft 11 by engaging a driver-eyelet attachment mechanism 99; and (ii) securing the eyelet 30 with an anchor 70 within a bone hole by disengaging the driver-eyelet attachment mechanism 99. The driver-eyelet attachment mechanism 99 may include one or more prongs 15 on the driver shaft 11 configured to mate with one or more corresponding apertures 35 on the eyelet 30. The prongs 15 are configured to flex in and out of the eyelet apertures 35. The anatomical tissue may be rotator cuff. The repair may be a Lisfranc repair.

Flexible strand 66 may be any flexible coupler and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strand 66 may have cross-sections of various forms and geometries, including round, oval, rectangular, or flat, among others, or combination of such forms and geometries. In some implementations, flexible strand 66 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible strand 66 may be braided or multi-filament suture such as FiberTape® suture tape (as disclosed in U.S. Pat. No. 7,892,256, the disclosure of which is incorporated in its entirety herewith). Flexible strand 66 may include elastic material. Flexible strand 66 may consist essentially of elastic suture.

Various regions or sections of flexible strand 66 may be coated and/or provided in different colors for easy manipulation during the surgical procedures. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A driver-eyelet attachment mechanism comprising:
a driver having a shaft with a longitudinal axis, a proximal end and a distal end, and a plurality of prongs extending away from the distal end and about parallel to the longitudinal axis of the shaft; and
an eyelet with a plurality of apertures configured to securely engage with and disengage from the plurality of prongs of the shaft, and with a suture passage configured to hold at least one flexible strand.

2. The driver-eyelet attachment mechanism of claim 1, wherein the plurality of prongs is configured to flex in and out of the plurality of apertures of the eyelet.

3. The driver-eyelet attachment mechanism of claim 2, wherein the plurality of prongs flex in when the eyelet is pushed onto the driver.

4. The driver-eyelet attachment mechanism of claim 3, wherein the plurality of prongs flex out when the eyelet is seated on the distal end of the driver.

5. The driver-eyelet attachment mechanism of claim 1, further comprising a cannulated fixation device with a body having an inner surface defining a cannulation extending axially through the body.

6. The driver-eyelet attachment mechanism of claim 5, wherein the driver extends through the cannulation of the body and is movable axially relative to the body.

7. The driver-eyelet attachment mechanism of claim 5, wherein, when the cannulated fixation device is pushed up against the eyelet, the plurality of prongs flex in and the eyelet is pushed off the distal end of the driver.

8. The driver-eyelet attachment mechanism of claim 5, wherein the cannulated fixation device is a swivel anchor.

9. The driver-eyelet attachment mechanism of claim 5, wherein the cannulated fixation device is an anchor, an interference plug, a screw, or an implant.

10. The driver-eyelet attachment mechanism of claim 1, wherein the at least one flexible strand is suture or suture tape.

11. The driver-eyelet attachment mechanism of claim 1, wherein the driver includes two prongs located symmetrically relative to the longitudinal axis of the shaft.

12. The driver-eyelet attachment mechanism of claim 11, wherein the two prongs are integrally formed with the shaft.

13. The driver-eyelet attachment mechanism of claim 11, wherein the shaft is solid and non-cannulated.

14. A surgical anchor assembly for tissue repairs, comprising:
an anchor with a cannulated anchor body and an outer surface having at least one fixation structure for securing the cannulated anchor body in a bone hole; and
an eyelet that is separable from the anchor, wherein the eyelet has an eyelet body with an aperture configured to hold at least one flexible strand, and wherein a most proximal end of the eyelet body contacts and abuts a most distal end of the cannulated anchor body when the surgical assembly is secured within tissue.

15. The surgical anchor assembly of claim 14, wherein the anchor is a swivel anchor.

16. The surgical anchor assembly of claim 14, wherein the eyelet body further comprises a plurality of openings configured to securely engage a plurality of flexible prongs of a driver shaft passing through the cannulated anchor body.

17. The surgical anchor assembly of claim 14, wherein the at least one flexible strand extends along the outer surface of the anchor when the surgical assembly is secured within hard tissue.

18. The surgical anchor assembly of claim 14, wherein the at least one fixation structure includes one or more threads.

19. The surgical anchor assembly of claim 14, wherein the surgical anchor assembly consists essentially of the anchor, the eyelet, and the at least one flexible strand.

20. A method of implanting a swivel anchor construct with a driver assembly, the swivel anchor construct comprising a fixation device and an eyelet that is separable from the fixation device, wherein the fixation device has an inner surface defining a cannulation, wherein the eyelet has an eyelet body with an aperture configured to hold at least one flexible strand, the driver assembly comprising a driver with a driver shaft terminating in a plurality of prongs, wherein the driver shaft extends through the cannulation of the fixation device so that the fixation device is movable axially relative to the driver shaft, the method comprising:
inserting the eyelet on the driver shaft; and
flexing the plurality of prongs inwardly while pushing the eyelet on.

21. The method of claim 20, further comprising:
flexing the plurality of prongs outwardly once the eyelet is fully inserted;
passing the at least one flexible strand through the aperture of the eyelet body;
advancing the swivel anchor construct and the at least one flexible strand to a desired depth in a bone hole;
advancing the cannulated fixation device distally into the bone hole so that a most distal end of the cannulated fixation device abuts and contacts a most proximal end of the eyelet; and
disengaging the eyelet from the plurality of prongs.

22. The method of claim 21, further comprising rotating the driver clockwise while the body is held against rotation to rotate the fixation device clockwise and advance the fixation device distally into the bone hole to engage the eyelet and to hold the eyelet and the at least one flexible strand in place in the bone hole.

23. The method of claim 21, further comprising removing the driver from the implanted swivel anchor construct.

24. The method of claim 20, wherein the fixation device is a cannulated anchor.
